(19) United States
(12) Patent Application Publication
(10) Pub. No.: US 2014/0343346 A1
(43) Pub. Date: Nov. 20, 2014

(54) CHARGED PARTICLE BEAM IRRADIATION DEVICE

(71) Applicant: SUMITOMO HEAVY INDUSTRIES, LTD., Tokyo (JP)

(72) Inventor: Satoru YAJIMA, Niihama-shi (JP)

(21) Appl. No.: 14/444,008

(22) Filed: Jul. 28, 2014

Related U.S. Application Data
(63) Continuation of application No. PCT/JP2012/052528, filed on Feb. 3, 2012.

(51) Int. Cl. A61N 5/10

(52) U.S. Cl. CPC A61N 5/1048; A61N 5/1078; A61N 2005/1094
USPC 600/1

(57) ABSTRACT

A charged particle beam irradiation device configured to irradiate a subject to be irradiated with a charged particle beam, including: an accelerator configured to accelerate charged particles and emit the charged particle beam; a gantry in which an irradiation unit for irradiating the subject with the charged particle beam is disposed; an irradiation table on which the subject is positioned; and a transportation line that includes an energy selection system for adjusting energy of the charged particle beam, and transport the charged particle beam to the irradiation unit from the accelerator. The transportation line is linearly formed up to the gantry from the accelerator, and at least a part of the energy selection system is disposed in the gantry, and a shield member configured to shield a radiation beam is provided between the energy selection system, which is disposed in the gantry, and the irradiation table.

### INCORPORATION BY REFERENCE

Priority is claimed to International Patent Application No. PCT/JP2012/052528, the entire content of each of which is incorporated herein by reference.

### BACKGROUND

1. Technical Field

The present invention relates to a charged particle beam irradiation device that irradiates a subject to be irradiated with a charged particle beam.

2. Description of the Related Art

In the past, a charged particle beam irradiation device disclosed in, for example, Pamphlet of International Publication No. WO 2011/036254 has been known as a charged particle beam irradiation device that is used in a radiation treatment or the like for a cancer. This charged particle beam irradiation device includes an accelerator that accelerates charged particles and emits a charged particle beam, an irradiation unit that irradiates a patient with the charged particle beam, a gantry in which the irradiation unit is disposed, and a transportation line that transports the charged particle beam emitted from the accelerator to the irradiation unit.

Further, the transportation line of the charged particle beam irradiation device includes an energy selection system that adjusts the energy of the charged particle beam emitted from the accelerator. In order to avoid a negative influence of a radiation beam, the energy selection system is generally disposed in a separate chamber (for example, an accelerator chamber in which the accelerator is disposed). A wall is interposed between the separate chamber and an irradiation chamber in which the gantry is disposed.

### SUMMARY

The invention provides a charged particle beam irradiation including an accelerator configured to accelerate charged particles and emit the charged particle beam, a gantry in which an irradiation unit for irradiating a subject to be irradiated with the charged particle beam is disposed, an irradiation table on which the subject to be irradiated is positioned, and a transportation line that includes an energy selection system for adjusting energy of the charged particle beam emitted from the accelerator and configured to transport the charged particle beam to the irradiation unit from the accelerator. The transportation line is linearly formed up to the gantry from the accelerator, and at least a part of the energy selection system is disposed in the gantry. A shield member configured to shield a radiation beam emitted toward the irradiation table is provided between the energy selection system, which is disposed in the gantry, and the irradiation table.

### BRIEF DESCRIPTION OF THE DRAWINGS

FIG. 1 is a schematic plan view showing a charged particle beam irradiation device according to one embodiment.

FIG. 2 is a schematic side view showing a state in which a gantry of FIG. 1 is rotated by an angle of 90°.

FIG. 3 is a schematic plan view showing a charged particle beam irradiation device according to an another embodiment.

FIG. 4 is a cross-sectional view taken along line IV-IV of FIG. 3.

FIG. 5 is a schematic plan view showing a charged particle beam irradiation device according to a further embodiment.

FIG. 6 is a schematic side view showing a state in which a gantry of FIG. 5 is rotated by an angle of 90°.

### DETAILED DESCRIPTION

Incidentally, it is strongly desirable to reduce the above-mentioned charged particle beam irradiation device in size in terms of the reduction of cost or the reduction of a site area. On the other hand, it is necessary to take appropriate measures in order to avoid a negative influence of a radiation beam, which is generated from the energy selection system or the like, on a patient.

Accordingly, it is desirable to provide a charged particle beam irradiation device that can be reduced in size and appropriately shield a radiation beam emitted toward a subject to be irradiated from an energy selection system.

According to the charged particle beam irradiation device, the transportation line is linearly formed up to the gantry from the accelerator. Accordingly, since it is possible to shorten the transportation line as compared to a case in which the transportation line is formed to be curved, it is possible to reduce the size of the device. Moreover, since at least a part of the energy selection system is disposed in the gantry in the charged particle beam irradiation device, it is possible to further shorten the transportation line as compared to the related art in which the entire energy selection system is disposed in a separate chamber that is separated from the irradiation chamber with a wall interposed therebetween. Accordingly, it is possible to further reduce the size of the device. Further, according to the charged particle beam irradiation device, the shield member is provided between the energy selection system, which is disposed in the gantry, and the irradiation table. Accordingly, the shield member can appropriately shield a radiation beam that is emitted toward the irradiation table from the energy selection system.

The shield member may be fixed to the gantry.

According to this structure, when the gantry is rotated, the shield member is also rotated integrally with the energy selection system. Accordingly, even though the shield member is small as compared to a case in which the shield member is formed separately from the gantry, the shield member can shield a radiation beam emitted toward the irradiation table. Therefore, according to this structure, it is advantageous to reduce the size and cost of the shield member.

The shield member may be provided so as to cover a portion of the energy selection system facing the irradiation table.

According to this structure, a radiation beam emitted from the energy selection system can be shielded before spreading toward the irradiation table. Accordingly, even though the shield member is small, it is possible to widely secure an area to which a radiation beam is not transmitted. This contributes to the improvement of the safety of a patient or a doctor and a maintenance worker.

Alternatively, the shield member may be installed separately from the gantry.

According to this structure, the gantry does not need to support the shield member as compared to a case in which the shield member is fixed to the gantry. Accordingly, it is advantageous in reducing the size and weight of the gantry.

The shield member may be provided so as to cover a portion of the irradiation table facing the energy selection system.

According to this structure, the shield member can appropriately and more reliably shield a radiation beam emitted toward the irradiation table from the energy selection system.

According to the embodiment of the invention, it is possible to reduce the size of the charged particle beam irradiation device and to shield a radiation beam emitted toward a subject to be irradiated from an energy selection system.

A charged particle beam irradiation device according to the embodiment of the invention will be described in detail below with reference to the drawings. Meanwhile, in the description of the drawings, the same elements are denoted by the same reference numerals and repeated description will be omitted.

### First Embodiment

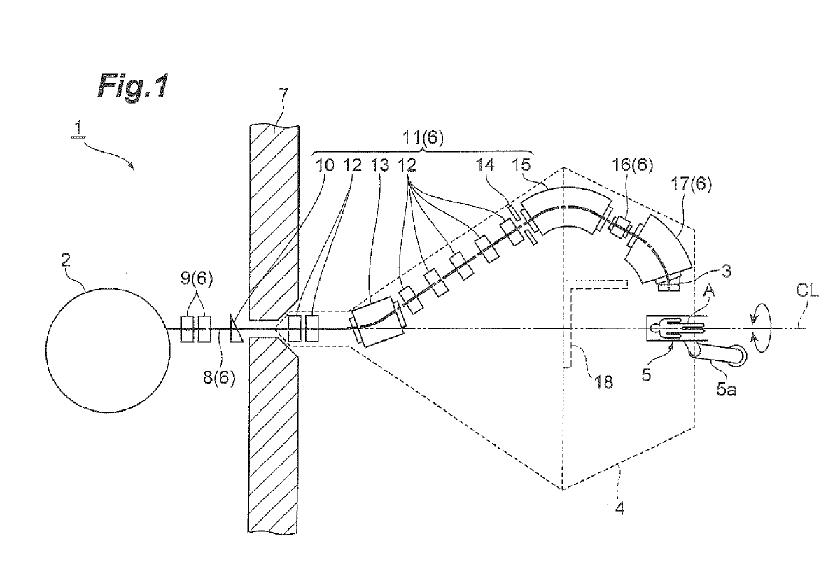

FIG. 1 is a schematic plan view showing a charged particle beam irradiation device 1 according to a first embodiment. As shown in FIG. 1, the charged particle beam irradiation device 1 is a device that is used in a radiation treatment for irradiating a tumor (a subject to be irradiated) of a patient A with a charged particle beam. The charged particle beam irradiation device 1 is accommodated in a building including a plurality of rooms.

The charged particle beam irradiation device 1 includes an accelerator 2 that accelerates charged particles and emits a charged particle beam, a gantry 4 in which an irradiation unit 3 for irradiating a tumor of a patient A with the charged particle beam is disposed, a treatment table (irradiation table) 5 on which the patient A is positioned, and a transportation line 6 that transports the charged particle beam emitted from the accelerator 2 to the irradiation unit 3.

The accelerator 2 emits a proton beam, a heavy particle (heavy ion) beam, or the like as the charged particle beam. For example, a cyclotron, a synchrotron, a synchrocyclotron, or a linear accelerator can be used as the accelerator 2. The accelerator 2 is disposed at a position that is separated from the gantry 4 with a wall 7 of the building interposed therebetween, and the wall 7 shields a radiation beam emitted from the accelerator 2. In the following description, a room in which the gantry 4 is disposed is referred to as an irradiation chamber and a room in which the accelerator 2 is disposed is referred to as an accelerator chamber.

The gantry 4 is adapted so as to be capable of rotating about a central axis CL by an angle of 360°, and is provided around the central axis CL so as to surround the treatment table 5. An irradiation unit 3 is mounted in the gantry 4 so as to irradiate the treatment table 5 with a charged particle beam. Since the irradiation unit 3 is rotated integrally with the gantry 4, the irradiation unit 3 can freely change an irradiation angle of the charged particle beam relative to the patient A positioned on the treatment table 5.

The treatment table 5 on which the patient A is positioned is movably supported by a robot arm 5a. The robot arm 5a moves the treatment table 5 in a horizontal direction and a vertical direction at the time of treatment, and disposes the treatment table 5 in the gantry 4. A root portion of the robot arm 5a is disposed outside the gantry 4, and is fixed to the floor of the building.

The transportation line 6 is formed to connect the accelerator 2 with the irradiation unit 3. The transportation line 6 is linearly formed up to the gantry 4 from the accelerator 2, and is connected to the irradiation unit 3 through the inside of the gantry 4. The transportation line 6 is formed so as to pass through the wall 7 that separates the accelerator chamber from the irradiation chamber, and linearly extends between the accelerator 2 and the gantry 4.

The transportation line 6 includes a vacuum duct 8 that forms a passage through which a charged particle beam passes. Two accelerator-side convergence magnets 9, which converge the diameter of the charged particle beam emitted from the accelerator 2, are disposed on the upstream side of the vacuum duct 8 (on the side of the vacuum duct 8 facing the accelerator 2). A degrader 10, which attenuates the energy of a charged particle beam, is disposed on the downstream side of the accelerator-side convergence magnets 9 (on the side of the accelerator-side convergence magnets 9 facing the irradiation unit 3). The accelerator-side convergence magnets 9 and the degrader 10 are disposed in the accelerator chamber in which the accelerator 2 is disposed.

The transportation line 6 includes an energy selection system (ESS) 11 that includes the degrader 10 as a component. The energy selection system 11 adjusts energy into desired energy according to a treatment plan by attenuating the charged particle beam that is emitted from the accelerator 2 and has constant energy. The energy selection system 11 selects the energy range of a charged particle beam that is transported by the transportation line 6 according to the treatment plan.

The energy selection system 11 includes gantry-side convergence magnets 12, a first deflection magnet 13, a slit 14, and a second deflection magnet 15 in addition to the degrader 10. All components of the energy selection system 11 except for the degrader 10 are disposed in the gantry 4.

The gantry-side convergence magnets 12 are convergence magnets disposed in the irradiation chamber, and the total number of the gantry-side convergence magnets 12 is seven. Two of the seven gantry-side convergence magnets 12 are disposed on one downstream side of the degrader 10 so as to be lined up, and the first deflection magnet 13 is disposed on the further downstream side of the two gantry-side convergence magnets 12. The other five gantry-side convergence magnets 12 are disposed on the downstream side of the first deflection magnet 13 so as to be lined up.

The first deflection magnet 13 is an electromagnet that deflects the traveling direction of a charged particle beam. The first deflection magnet 13 deflects the charged particle beam, which has linearly traveled along the central axis CL, so as to incline the charged particle beam to the outside of the gantry 4.

The slit 14 is disposed on the downstream side of the five gantry-side convergence magnets 12 that are lined up. The slit 14 selects the energy of the charged particle beam by shielding apart of the charged particle beam that passes through the slit 14. The second deflection magnet 15 is disposed on the downstream side of the slit 14. The second deflection magnet 15 deflects the charged particle beam, which has traveled along the outside of the gantry 4, in a direction in which the charged particle beam approaches the central axis CL.

A scanning magnet 16 is disposed on the downstream side of the second deflection magnet 15. The scanning magnet 16 controls the scanning of the charged particle beam according to the treatment plan. A third deflection magnet 17 is disposed on the downstream side of the scanning magnet 16. The third deflection magnet 17 deflects a charged particle beam toward the irradiation unit 3. The scanning magnet 16 and the third deflection magnet 17 are also members that form the transportation line 6.

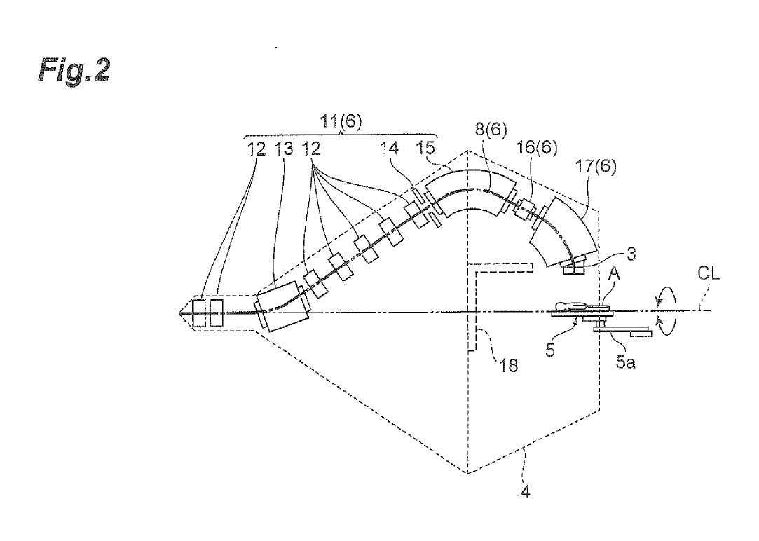

FIG. 2 is a schematic side view showing a state in which the gantry 4 is rotated by an angle of 90°. In FIG. 2, the gantry 4 is rotated so that the irradiation unit 3 is positioned above the patient A. As shown in FIGS. 1 and 2, the charged particle beam irradiation device 1 includes an L-shaped shield member 18 that shields a radiation beam emitted toward the patient A (the treatment table 5).

Since the L-shaped shield member 18 is fixed to the gantry 4, the L-shaped shield member 18 is rotated integrally with the gantry 4. For this reason, a positional relationship between the energy selection system 11 and the shield member 18, which are disposed in the gantry 4, is not changed by the rotation of the gantry 4. The L-shaped shield member 18 has a sufficient width to shield a radiation beam that is emitted from the energy selection system 11. The shield member 18 is made of, for example, lead, iron, or tungsten.

According to the charged particle beam irradiation device 1 of the above-mentioned first embodiment, the transportation line 6 is linearly formed up to the gantry 4 from the accelerator 2. Accordingly, since it is possible to shorten the transportation line 6 as compared to a case in which the transportation line 6 is formed to be curved, it is possible to reduce the size of the device. Moreover, since a part of the energy selection system 11 is disposed in the gantry 4 in the charged particle beam irradiation device 1, it is possible to further shorten the transportation line 6 as compared to the related art in which the entire energy selection system 11 is disposed in an accelerator chamber. Accordingly, it is possible to further reduce the size of the device. Further, according to the charged particle beam irradiation device 1, the shield member 18 is provided between the energy selection system 11 and the treatment table 5 that are disposed in the gantry 4. Accordingly, the shield member 18 can appropriately shield a radiation beam that is emitted toward the patient A positioned on the treatment table 5 from the energy selection system 11.

Furthermore, according to the charged particle beam irradiation device 1, when the gantry 4 is rotated, the shield member 18 is also rotated integrally with the energy selection system 11. Accordingly, even though the shield member 18 is small as compared to a case in which the shield member 18 is formed separately from the gantry 4, the shield member 18 can shield a radiation beam emitted toward the treatment table 5. Therefore, according to the charged particle beam irradiation device 1, it is possible to reduce the size and cost of the shield member 18.

### Second Embodiment

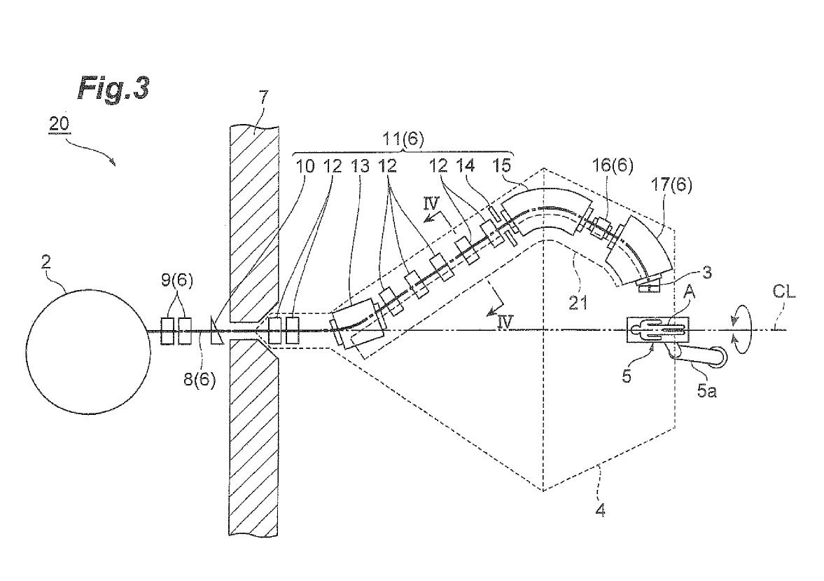

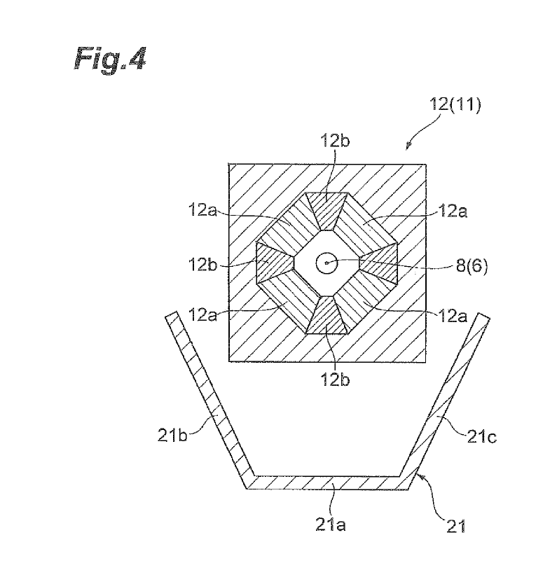

FIG. 3 is a schematic plan view showing a charged particle beam irradiation device 20 according to a second embodiment. FIG. 4 is a cross-sectional view taken along line IV-IV of FIG. 3.

As shown in FIGS. 3 and 4, the charged particle beam irradiation device 20 according to the second embodiment is different from the charged particle beam irradiation device 1 according to the first embodiment in terms of the shape and the position of a shield member.

Specifically, the charged particle beam irradiation device 20 according to the second embodiment includes a gutter-shaped shield member 21 that extends along the transportation line 6 that is disposed in the gantry 4. The gutter-shaped shield member 21 is formed so as to be curved along the transportation line 6, which is disposed in the gantry 4, as a whole. Meanwhile, the gutter-shaped shield member 21 may not be formed along the entire transportation line 6, and may be formed along only the energy selection system 11.

As shown in FIG. 4, the gutter-shaped shield member 21 is formed so as to cover a portion, which is close to the treatment table 5, of the energy selection system 11 that is disposed in the gantry 4. Specifically, the shield member 21 includes a bottom 21a that is positioned close to the treatment table 5 when seen from the energy selection system 11 (the convergence magnet 12) and a pair of side walls 21b and 21c that are positioned so that the energy selection system 11 (the convergence magnet 12) is interposed between the side walls 21b and 21c, and is formed in the shape of a gutter. Meanwhile, reference numerals 12a shown in FIG. 4 denote magnetic poles and reference numerals 12b denote coils.

According to the charged particle beam irradiation device 20 of the above-mentioned second embodiment, a radiation beam emitted from the energy selection system 11 can be shielded before spreading toward the treatment table 5. Accordingly, even though the shield member 21 is small, it is possible to secure a wide area to which a radiation beam is not transmitted. Therefore, according to the charged particle beam irradiation device 20, it is possible to improve the safety of a patient or a doctor and a maintenance worker.

### Third Embodiment

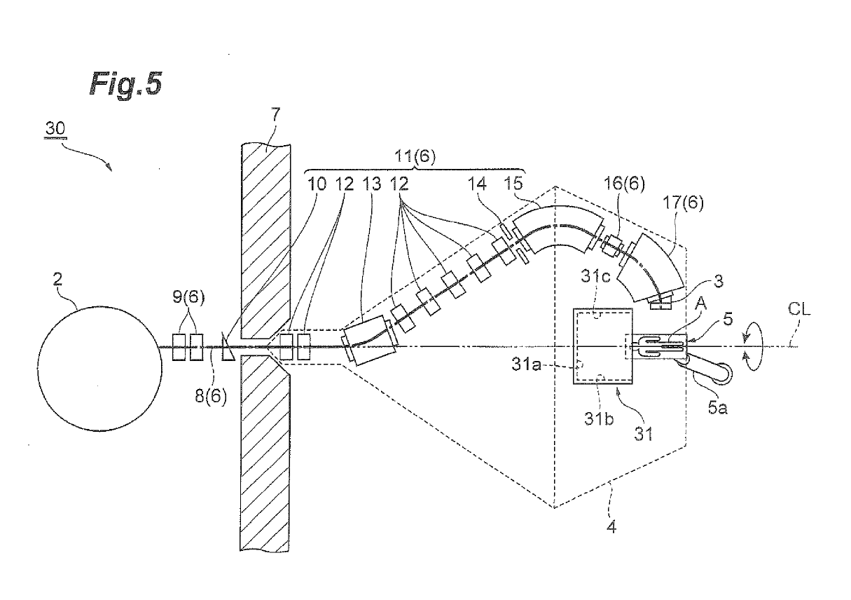

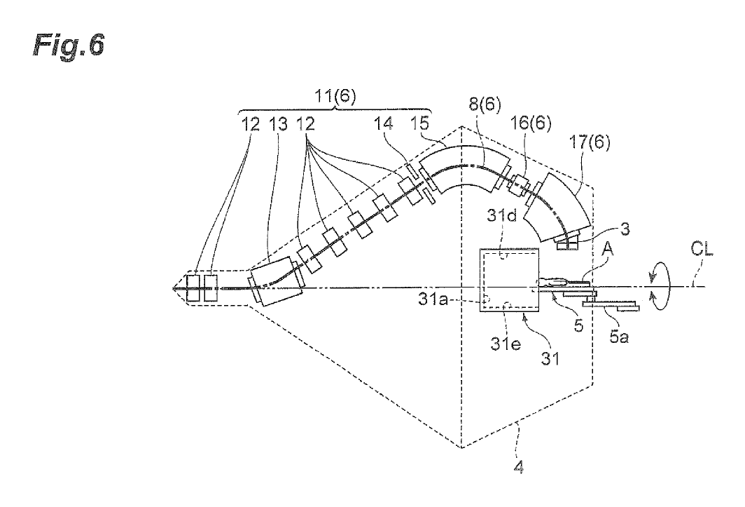

FIG. 5 is a schematic plan view showing a charged particle beam irradiation device 30 according to a third embodiment. FIG. 6 is a schematic side view showing a state in which a gantry 4 of FIG. 5 is rotated by an angle of 90°.

As shown in FIGS. 5 and 6, the charged particle beam irradiation device 30 according to the third embodiment is different from the charged particle beam irradiation device 1 according to the first embodiment in terms of the shape and the position of a shield member and the support structure of the shield member.

Specifically, the charged particle beam irradiation device 30 according to the third embodiment includes a shield member 31 that is installed separately from the gantry 4. Since the shield member 31 is separated from the gantry 4 that can be rotated by an angle of 360°, the position of the shield member 31 is not changed by the rotation of the gantry 4. The shield member 31 is fixed to a building that accommodates the charged particle beam irradiation device 30. The shield member 31 is connected to the building by a connection member (not shown) that extends along the central axis CL from the front of the charged particle beam irradiation device 30. The shield member 31 is formed so as to cover a portion of the treatment table 5 facing the energy selection system 11. The shield member 31 is formed in the shape of a box that is opened toward the front side of the gantry 4 (the side of the gantry 4 facing the treatment table 5), and a space in which the treatment table 5 moves is formed in the shield member 31. Specifically, the shield member 31 includes a side wall 31a that is positioned on the back side when seen from the front of the gantry 4, side walls 31b and 31c that are positioned on the left and right sides of the treatment table 5, a ceiling 31d, and a floor 31e, and is formed in the shape of a box. The shield member 31 is formed so as to reliably shield a radiation beam emitted toward a patient A, who is positioned on the treatment table 5, while sufficiently securing a space in which the treatment table 5 moves.

According to the charged particle beam irradiation device 30 of the above-mentioned third embodiment, the gantry 4 does not need to support the shield member 31 as compared to a case in which the shield member is fixed to the gantry 4. Accordingly, it is advantageous in reducing the size and weight of the gantry 4. Moreover, since the shield member 31 is formed so as to cover a portion of the treatment table 5 facing the energy selection system 11 in the charged particle beam irradiation device 30, the shield member 31 can more reliably shield a radiation beam emitted toward the patient A, who is positioned on the treatment table 5, from the energy selection system 11.

The invention is not limited to the above-mentioned embodiments. For example, the position and the shape of the shield member are not limited to the above-mentioned the positions and the shapes of the shield members, and the shield member needs only to be capable of shielding a radiation beam emitted toward the patient.

Further, when the shield member is fixed to the gantry 4, the shield member may function as a frame of the gantry 4. That is, the shield member may be used as a frame of the gantry 4. Furthermore, the shield member is not limited to a member necessarily having stiffness, may be a sheet-like member, and may be formed of a member that has the shape of a plurality of sheets.

Meanwhile, the embodiments of the invention can be effectively applied regardless of whether a charged particle beam irradiation method is a wobbler type method or a scanning type method.

Moreover, the gantry 4 is not limited to a structure that can be rotated about the central axis CL by an angle of 360°, and may have a structure that can oscillate by an angle smaller than 360° (for example, 200°). In this structure, the floor of a building can be formed in a space that is present around the central axis CL and outside a moving range of the gantry 4. In this case, the shield member 31 according to the third embodiment may be directly formed on the floor of the building around the central axis CL. Further, a root portion of the robot arm 5a can also be provided on this floor. Furthermore, the floor 31e may be unnecessary.

It should be understood that the invention is not limited to the above-described embodiment, but may be modified into various forms on the basis of the spirit of the invention. Additionally, the modifications are included in the scope of the invention.

What is claimed is:

1. A charged particle beam irradiation device comprising:
an accelerator configured to accelerate charged particles and emit the charged particle beam;
a gantry in which an irradiation unit for irradiating a subject to be irradiated with the charged particle beam is disposed;
an irradiation table on which the subject to be irradiation is positioned; and
a transportation line that includes an energy selection system for adjusting energy of the charged particle beam emitted from the accelerator, and configured to transport the charged particle beam to the irradiation unit from the accelerator,
wherein the transportation line is linearly formed up to the gantry from the accelerator, and at least a part of the energy selection system is disposed in the gantry, and
a shield member configured to shield a radiation beam emitted toward the irradiation table is provided between the energy selection system, which is disposed in the gantry, and the irradiation table.

2. The charged particle beam irradiation device according to claim 1,
wherein the shield member is fixed to the gantry.

3. The charged particle beam irradiation device according to claim 2,
wherein the shield member is provided so as to cover a portion of the energy selection system facing the irradiation table.

4. The charged particle beam irradiation device according to claim 1,
wherein the shield member is installed separately from the gantry.

5. The charged particle beam irradiation device according to claim 4,
wherein the shield member is provided so as to cover a portion of the irradiation table facing the energy selection system.
